(19) United States
(12) Patent Application Publication
(10) Pub. No.: US 2024/0252723 A1
(43) Pub. Date: Aug. 1, 2024

(54) INDUCED PLURIPOTENT STEM CELL (IPSC) DERIVED MULTI-RIBBON BIODEGRADABLE GEL FOR OUTER RETINAL REPLACEMENT

(71) Applicant: Alcon Inc., Fribourg (CH)

(72) Inventor: Steven T. Charles, Memphis, TN (US)

(21) Appl. No.: 18/422,182

(22) Filed: Jan. 25, 2024

Related U.S. Application Data
(60) Provisional application No. 63/481,809, filed on Jan. 27, 2023.

(51) Int. Cl. A61L 27/38; A61K 9/00; A61K 9/06; A61K 35/30; A61K 47/34; A61L 27/18; A61L 27/36; A61L 27/44; A61L 27/58

(52) U.S. Cl. CPC A61L 27/3804; A61K 9/0051; A61K 9/06; A61K 35/30; A61K 47/34; A61L 27/18; A61L 27/3604; A61L 27/44; A61L 27/58; A61L 2430/16

(57) ABSTRACT

Described herein is a method for replacing retinal tissue with retinal cell-embedded biodegradable matrix ribbons. The biodegradable matrix ribbons can be provided as a sheet and subsequently cut into ribbons. Alternatively, the biodegradable matrix ribbons can be constructed in ribbon form. The thin ribbons can then be implanted into a patient's eye through small scleral and/or retinal incisions. Once inside the ocular space, the ribbons can be positioned at a target retinal space in a confluent fashion in order to provide an area of biodegradable matrix having retinal cells embedded within and on outer surfaces thereof.

### BACKGROUND

Retinal degenerative diseases are one of the leading causes of irreversible blindness worldwide, and treatment options are limited. Common to all retinal degenerative diseases is the damage or loss of photoreceptor cells of the retina. The photoreceptor cells are the light sensing cells of the retina, the delicate layer that lines the back of the eye. Photoreceptor cell loss can occur as a consequence of separation from the underlying retinal pigment epithelium (RPE) and/or apoptosis. In any case, photoreceptor loss leads to progressive visual impairment.

One of the most common retinal degenerative diseases is age-related macular degeneration (AMD). AMD is a deterioration of the macula, or the central part of the retina responsible for central, high-resolution, color vision. AMD leads to a substantial reduction in sharpness of vision, and is the leading cause of visual deterioration in people over the age of 60.

Most current treatments can reduce the rate of disease progression; however, they generally fail to completely stop photoreceptor cell loss. These treatments include antioxidant supplementation, complement inhibition using intravitreal injections, lifestyle and dietary modification, intravitreal antiangiogenic therapy, gene therapy, and implanted visual prostheses. Over the last decade, human pluripotent stem cells, such as embryonic stem cells and induced pluripotent stem cells, have gained attention as a potential option for treating retinal degenerative diseases like AMD. Embryonic stem cells (ESCs) are pluripotent stem cells derived from the inner cell mass of an early-stage pre-implantation embryo. Induced pluripotent stem cells (iPSCs), on the other hand, do not come from embryos; rather, they are derived from differentiated somatic cells. Pluripotent stem cells can be used for a wide range of applications because they are able to self-renew and give rise to all the body's cell lineages.

Attempts have been made to address retinal degenerative disorders by implanting induced pluripotent stem cells into the eye. However, these previous treatment methods are associated with certain drawbacks. For example, methods that involve infusion of iPSC suspensions suffer from low engraftment rates. Further, infusions of suspensions have been known to leak to the anterior retinal surface and lead to retinal surface scarring as well as retinal detachment. Some other methods have involved the use of scaffolds for securing the iPSCs in place and preventing cellular loss. However, many scaffold materials, including natural and naturally derived scaffold materials like silks, alginates, polyesters, and parylene lead to chronic inflammation.

Therefore, there remains a need for methods of treating retinal degenerative diseases that can induce new cell growth and potentially reverse cell loss.

### SUMMARY

The present disclosure generally relates to compositions, devices, and methods for replacing retinal tissue with a retinal cell-embedded biodegradable matrix.

In certain embodiments, a method of implanting retinal cells into a patient's eye is provided. The method comprises: combining retinal cells and a biodegradable polymer scaffold to form a gel; aspirating at least a portion of the gel into a cannula; forming an incision in a sclera of the patient's eye; inserting the cannula through the incision in the sclera of the eye; and depositing the gel in a target area of the patient's eye.

In certain embodiments, a method of generating gelatinous biodegradable ribbons for retinal replacement is provided. The method comprises: providing a receptacle comprising a space and at least one partition member; delivering a solution of a biodegradable polymer to the space; delivering retinal cells to the space; and culturing in vitro for a period of time said retinal cells and said biodegradable polymer to afford gelatinous ribbons comprising a biodegradable polymer matrix and said retinal cells.

In certain embodiments, a method of implanting retinal cells into a patient's eye is provided. The method comprises: aspirating a gelatinous biodegradable ribbon into a cannula, wherein the gelatinous biodegradable ribbon comprises a biodegradable polymer matrix and retinal cells; forming an incision in a sclera of the patient's eye; inserting the cannula through the incision in the sclera of the eye; and depositing the gelatinous biodegradable ribbon in a target area of the patient's eye.

### BRIEF DESCRIPTION OF THE DRAWINGS

FIG. 1 is a schematic illustration of a culturing tray for culturing retinal cells within biodegradable polymer scaffolding, according to some embodiments herein.

FIG. 2 is a schematic illustration of a partitioned culturing tray for culturing retinal cells within partitioned ribbons of biodegradable polymer scaffolding, according to some embodiments herein.

FIG. 3 is a schematic of a retinal cell-embedded biodegradable polymer ribbon that includes visual indicators for identifying ribbon ends, according to some embodiments herein.

FIG. 4 is a schematic cross-sectional illustration of an applicator device having a cannula with a rectangular cross-sectional area, according to some embodiments herein.

FIG. 5 is a schematic of a proportional pedal controller that can be used in conjunction with a cannula to aspirate and deposit retinal cell-embedded biodegradable polymer matrix ribbons, according to some embodiments herein.

FIGS. 6A-6G schematically illustrate various operations of a method of performing retinal cell replacement, according to some embodiments herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The retina is the light sensing component of the eye that relays information to the occipital cortex of the brain via the optic nerve. The retina is made of layers having an ordered structure. The outer photoreceptors layer plays a role in light perception and the phototransduction. The retinal pigment epithelium (RPE) layer is located between the photoreceptor layer and the underlying pentalaminar Bruch's membrane. The retinal pigment epithelium layer, together with the photoreceptor layers, acts as functional unit, and its optimal functioning is critical to sight. Loss of the integrity of this unit, promotes degeneration of the photoreceptors.

Retinal degeneration occurs in several forms, such as retinitis pigmentosa, macular dystrophy, and non-neovascular age related macular degeneration referred to as geographic atrophy. Age related macular degeneration is associated with photoreceptor degradation; however, the inner retina remains intact. This multifactorial physiology makes photoreceptor replacement a viable therapy.

Surgical options to treat retinal degeneration can substitute affected retinal layers, replace lost or injured retinal components, and/or reestablish the interaction between retinal pigment epithelium cells and photoreceptors. Embryonic stem cells and existing pluripotent stem cell lines have been used as cell sources for retinal replacement; however, the use of these cells requires that the patient be transiently immune-suppressed. Further, cellular suspensions of retinal pigment epithelium have typically been used to replace affected retinal layers. Yet, these methods often suffer from poor engraftment. Even further, the scleral incision size is typically around 3.5 mm, and implanted cellular suspensions can leak out through this relatively large incision. Relatively large retinotomies can also lead to bleeding and retinal detachment. Some attempts at retinal replacement surgeries have employed the use of scaffolds to prevent cell leakage, but most biocompatible scaffold materials are non-biodegradable and lead to chronic inflammation.

The embodiments described herein provide compositions and methods that address the problems discussed above. Such compositions and methods may be utilized for treating retinal degenerative diseases like age-related macular degeneration. The methods involve the deposition of scaffold-embedded retinal cells (e.g., polarized RPE cells and polarized photoreceptor progenitor cells) in the form of ribbons (i.e., strips). The ribbons can be provided in the form of a semi-rigid and flexible gel. Multiple ribbons can be deposited on a patient's retinal surface in adjoining relationships such that a contiguous plank area of scaffold-supported cells is assembled.

Similar to a stack bond wooden flooring or masonry pattern (e.g., tiling or laid bricks), the ribbons can be deposited side-by-side such that one ribbon length abuts against and is contiguous with an adjacent ribbon length. The scaffold within each ribbon provides a framework within and on which cells, and optionally cell nutrients and/or pharmacological agents, can be supported.

Methods can include the use of induced pluripotent stem cells as a source for replacement retinal cells. The induced pluripotent stem cells can be prepared in a relatively short (e.g., 100 day) period, and do not require that the patient be immune-suppressed. The replacement retinal cells derived from the induced pluripotent stem cells can be provided on a biodegradable scaffold. The use of a biodegradable scaffold averts the chronic inflammation issues that arise when using non-biodegradable scaffolds. The use of a scaffolding material also helps maintain the cells in the desired location and prevents leakage out of the eye.

Additionally, the cell-loaded, biodegradable scaffold can be provided in the form of multiple thin ribbons (i.e., strips). Because thin ribbons are employed, the scleral incision size required for passing the cell-loaded scaffolding through the eyewall is significantly smaller than the customary 3.5 mm (millimeters) sclerotomy size. For example, in certain embodiments, a sclerotomy having a maximum dimension of less than 1 mm, corresponding to less than a 23 gauge or 25 gauge cannula, may be utilized to introduce the ribbons into the eye. Similarly, in examples where a target area is within a subretinal space, the retinotomy incision size required for passing the scaffolding to the subretinal space is reduced. The relatively small size of the sclerotomy and/or retinotomy enabled by such ribbons reduces the risk of hypotony and retinal cell reflux to anterior retinal surfaces.

A plurality of thin ribbons can be inserted into the eye and assembled in a target area within the eye. Multiple ribbons can be deposited under the retina in an adjoining manner such that a contiguous, multi-strip area of scaffold-supported cells is assembled. Similar to a stack bond wooden flooring or masonry pattern, the ribbons can be assembled confluently, with side-by-side contact such that one strip length abuts against and is contiguous with an adjacent strip length. The scaffold within each strip provides a framework within and on which cells, and optionally cell nutrients, pharmacological agents, and/or visual indicators can be supported. The in situ assembly of multiple thin ribbons allows for deposition of a large, modular area of retinal cells. The additional coverage provided by the larger area will correspond to larger central visual field improvement, without making a larger retinotomy or scleral incision.

Accordingly, some embodiments of the present disclosure are directed to a method of implanting retinal cells into a patient's eye. In some embodiments, the method comprises combining retinal cells and a biodegradable polymer scaffold to form a gel, aspirating at least a portion of the gel into a cannula of an applicator device, forming an incision in a sclera of the patient's eye, inserting the cannula of the applicator device through the incision in the sclera of the eye, and depositing the gel in a target area of the patient's eye. In some embodiments, the target area comprises the retina. In some embodiments, the method further comprises combining at least one pharmacological agent, at least one cell nutrient, at least one visual indicator, or a combination thereof with the retinal cells and a biodegradable polymer scaffold. In some embodiments, the target area is a sub-retinal space in the patient's eye. In such embodiments, the method further comprises forming one or more incisions in the retina of the patient's eye, progressively inserting the cannula through each of the one or more incisions in the retina of the eye, and depositing a plurality of gel portions in a target area of the patient's eye. In some embodiments, the patient has been diagnosed with a retinal degenerative disease and/or is in need of a retinal transplant or replacement.

Some embodiments of the present disclosure are directed to a method of generating gelatinous biodegradable ribbons for retinal replacement. In some embodiments, the method comprises providing a receptacle comprising a space and at least one partition member, delivering a solution of a biodegradable polymer to the space, delivering retinal cells to the space, and culturing in vitro, for a period of time, said retinal cells and said biodegradable polymer to form gelatinous ribbons comprising a biodegradable polymer matrix and retinal cells. In some embodiments, the method of generating gelatinous biodegradable ribbons for retinal replacement further comprises delivering to the space at least one pharmacological agent, at least one cell nutrient, at least one visual indicator, or a combination thereof. In some embodiments, the ribbon further comprises at least one pharmacological agent, at least one cell nutrient, at least one visual indicator, or a combination thereof.

Some embodiments of the present disclosure are directed to a method of implanting retinal cells into a patient's eye, wherein the retinal cells are provided in a gelatinous biodegradable ribbon. In some embodiments, the method comprises aspirating a gelatinous biodegradable ribbon into a cannula of an applicator device, wherein the gelatinous biodegradable ribbon comprises a biodegradable polymer matrix and retinal cells, forming an incision in a sclera of the patient's eye, inserting the cannula of the applicator device through the incision in the sclera of the eye, and depositing the gelatinous biodegradable ribbon in a target area of the patient's eye. In some embodiments, the target area of the patient's eye is a sub-retinal space of the patient's eye. In such embodiments, the method further comprises forming one or more incisions in the retina of the patient's eye, forming a bleb under the retina, inserting and positioning the cannula at a far side of the bleb, and depositing a plurality of ribbons in a target area of the patient's eye as the cannula is withdrawn from the bleb. In some embodiments, the ribbons are deposited confluently in the target area of the patient's eye.

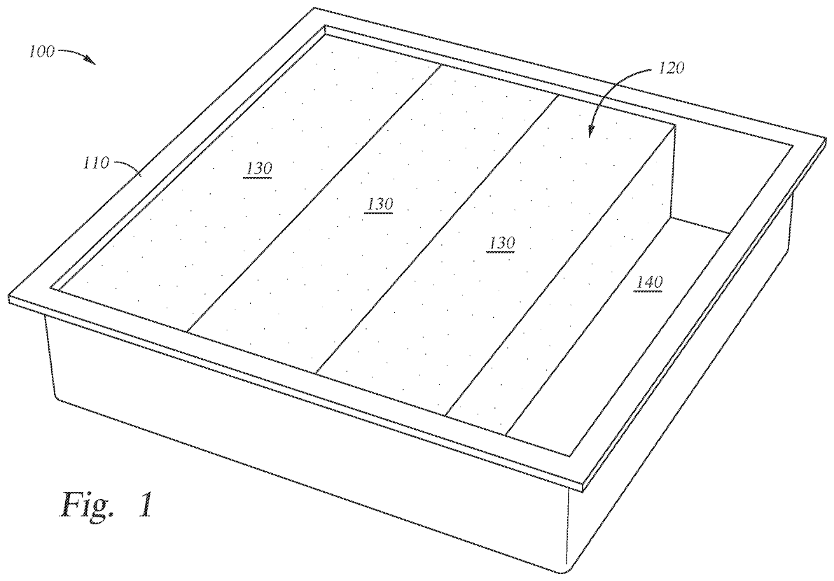

Referring to FIG. 1, a culture tray 110 for culturing retinal cells and biodegradable polymer is depicted. Within an interior volume of culture tray 110 is a matrix 120 that includes a biodegradable polymer and retinal cells. The biodegradable polymer serves as a scaffold that provides a framework within and on which the retinal cells, and optionally cell nutrients and/or pharmacological agents, can be supported. The matrix 120 is cut into a plurality of ribbons 130. One of the plurality of ribbons 130 has been removed from culture tray 110, leaving an empty space corresponding to empty tray volume 140.

In some embodiments, the biodegradable polymer comprises a material selected from a group consisting of poly(lactic-co-glycolic acid) (PLGA), collagen, gelatin, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatite, calcium phosphate, and combinations thereof. In some embodiments, the biodegradable polymer comprises a transparent or translucent material. In some embodiments, the biodegradable polymer comprises a pigmented material.

In some embodiments, the retinal cells comprise differentiated cells, progenitor cells, precursor cells, or a combination thereof. The differentiated cells, progenitor cells, precursor cells, or combination thereof can include retinal pigment epithelial (RPE) cells, rod cells, cone cells, horizontal cells, bipolar cells, amacrine cells, retinal ganglion cells, or a combination thereof. In some embodiments, the retinal cells comprise differentiated retinal pigment epithelial cells. In some embodiments, the retinal cells comprise photoreceptor progenitor cells. In some embodiments, the photoreceptor progenitor cells comprise rod progenitor cells, cone progenitor cells, or a combination thereof. In some embodiments, the retinal cells comprise a combination of differentiated retinal pigment epithelial cells, rod progenitor cells, and cone progenitor cells.

In some embodiments, the matrix 120 and thus, each ribbon 130, comprises a monolayer of differentiated cells, such as a monolayer of differentiated RPE cells. In some embodiments, the matrix 120 and each ribbon 130 comprise a bilayer of differentiated cells, such as a bilayer of differentiated RPE cells. In some embodiments, in addition to a monolayer or bilayer of differentiated cells, the matrix 120 and each ribbon 130 comprise a monolayer of progenitor cells, such as a monolayer of retinal photoreceptor progenitor cells. In such embodiments, the differentiated cells may have a basal polarity, and the progenitor cells may have an apical polarity. Differentiated RPE cells, photoreceptor cells, and their progenitors and precursors, can be implanted and positioned within a patient's target retinal space using any appropriate apicobasal polarity.

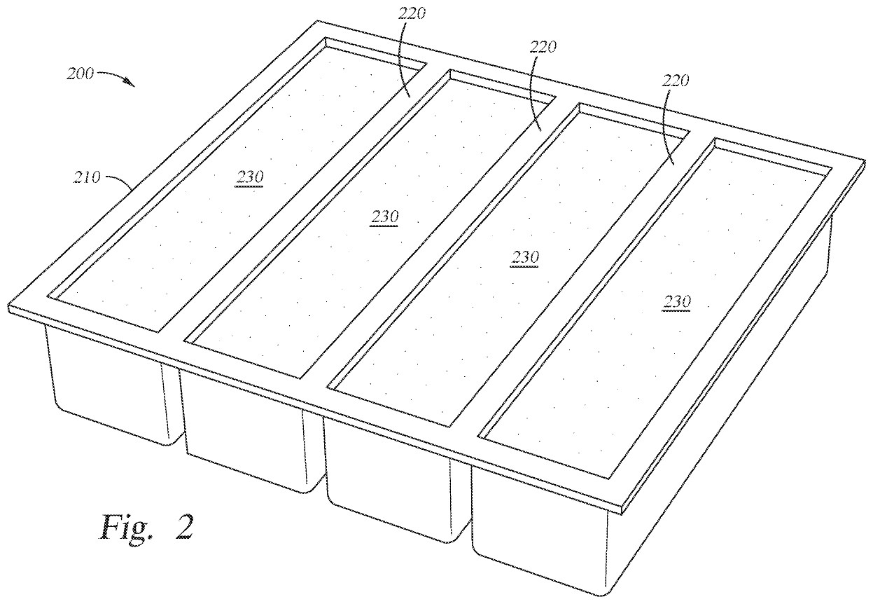

FIG. 2 depicts a partitioned receptacle 200, according to certain embodiments of the present disclosure. The partitioned receptacle 200 includes a space or volume 210 within which biodegradable polymer and retinal cells can be combined. Within receptacle space 210 are one or more partitions 220 that divide the receptacle space into a plurality of sub-spaces 230. In some embodiments, the one or more partitions 220 comprise substantially vertical walls. In some embodiments, the sub-spaces 230 formed by partitions 220 comprise elongated cross-sectional areas (e.g., morphologies). In some embodiments, the sub-spaces 230 comprise substantially rectangular cross-sectional areas. In some embodiments, the sub-spaces 230 are cuboid-shaped and have rectangular-shapes as viewed from above, such that a sub-space length dimension is greater than a sub-space width dimension.

In some embodiments, a receptacle 200 with an n integer number of partitions 220 will include an n+1 integer number of sub-spaces. For example, in the embodiment depicted in FIG. 2, the receptacle 200 includes three (n) partitions 220 and four (n+1) sub-spaces 230. The receptacle 200, receptacle space 210, partitions 220, and sub-spaces 230 are designed to contain a combination of biodegradable polymer and retinal cells. The receptacle 200 can contain the combination of biodegradable polymer and retinal cells during a culturing period where the retinal cells are allowed to grow and multiply. Cell nutrients, pharmacological agents, and/or visual indicators can be added to the combination of biodegradable polymer and retinal cells in the receptacle 200. The combination of biodegradable polymer and retinal cells give rise to gelatinous ribbons that include the retinal cells embedded within and on the biodegradable polymer, which serves as a scaffold for cell support. A visual indicator can be added to the ribbon to indicate the location of ribbon ends, for example. The visual indicator can assist a practitioner in identifying terminal ribbon positions when positioning the ribbons during surgery. The visual indicator can be a physical feature in the ribbon, or any dye or pigment known to those of skill in the art. An exemplary, non-limiting visual indicator is sodium fluorescein.

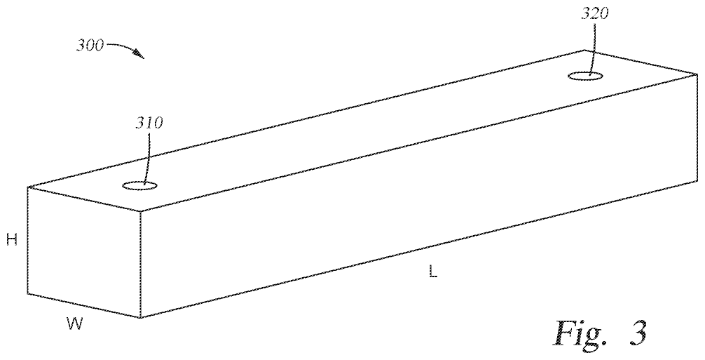

FIG. 3 depicts an exemplary ribbon 300 that includes a biodegradable polymer and retinal cells, according to certain embodiments of the present disclosure. The biodegradable polymer provides a semi-rigid framework and serves as a scaffold for supporting the retinal cells. The ribbon 300 has a height h, a width w, and a length l. The height h, width w, and/or length l may be between about 0.1 mm and about 1 mm, such as between about 0.2 mm and about 0.8 mm, such as between about 0.3 mm and about 0.7 mm, such as between about 0.4 mm and about 0.6 mm, such as about 0.5 mm. In certain embodiments, the height h, width w, and/or length l are between about 0.1 mm and about 0.9 mm, between about 0.1 mm and about 0.8 mm, between about 0.1 mm and about 0.7 mm, between about 0.1 mm and about 0.6 mm, between about 0.1 mm and about 0.5 mm, between about 0.1 mm and about 0.4 mm, between about 0.1 mm and about 0.3 mm, or between about 0.1 mm and about 0.2 mm. In certain embodiments, the height h, width w, and/or length l are between about 0.2 mm and about 1 mm, between about 0.2 mm and about 0.9 mm, between about 0.2 mm and about 0.7 mm, between about 0.2 mm and about 0.6 mm, between about 0.2 mm and about 0.5 mm, between about 0.2 mm and about 0.4 mm, or between about 0.2 mm and about 0.3 mm.

In certain embodiments, the height h, width w, and/or length l are between about 0.3 mm and about 1 mm, between about 0.3 mm and about 0.9 mm, between about 0.3 mm and about 0.8 mm, between about 0.3 mm and about 0.6 mm, between about 0.3 mm and about 0.5 mm, or between about 0.3 mm and about 0.4 mm. In certain embodiments, the height h, width w, and/or length l are between about 0.4 mm and about 1 mm, between about 0.4 mm and about 0.9 mm, between about 0.4 mm and about 0.8 mm, between about 0.4 mm and about 0.7 mm, or between about 0.4 mm and about 0.5 mm. In certain embodiments, the height h, width w, and/or length l are between about 0.5 mm and about 1 mm, between about 0.5 mm and about 0.9 mm, between about 0.5 mm and about 0.8 mm, between about 0.5 mm and about 0.7 mm, or between about 0.5 mm and about 0.6 mm.

In certain embodiments, the height h, width w, and/or length l are between about 0.6 mm and about 1 mm, between about 0.6 mm and about 0.9 mm, between about 0.6 mm and about 0.8 mm, or between about 0.6 mm and about 0.7 mm. In certain embodiments, the height h, width w, and/or length l are between about 0.7 mm and about 1 mm, between about 0.7 mm and about 0.9 mm, or between about 0.7 mm and about 0.8 mm. In certain embodiments, the height h, width w, and/or length l are between about 0.8 mm and about 1 mm, or between about 0.8 mm and about 0.9 mm. In certain embodiments, the height h, width w, and/or length l are between about 0.9 mm and about 1 mm.

In some embodiments, the ribbon 300 has dimensions based on a size of an incision in a sclera and/or retina of an eye. For example, in some embodiments, the ribbon 300 has lateral dimensions that facilitate a smaller incision in the sclera for introducing the ribbon 300 into the eye. In some embodiments, a plurality of ribbons 300 to be applied to a target area have collective dimensions equal or substantially equal to a size of a visual field to be restored.

The exemplary ribbon 300 depicted in FIG. 3 has a cuboid shape, with a length l being greater than a width w that has a rectangular face (e.g., a rectangular cross section) as viewed from above; however, other shapes are contemplated, including three-dimensional shapes with square, diamond, rounded, and triangular faces, for example. The ribbon 300 includes optional visual indicators 310 and 320 that are located near ribbon ends. Visual indicators 310 and 320 can assist the practitioner in identifying the ribbon ends and placement of the ribbons 300 during surgery. In some embodiments, a plurality of ribbons 300 placed in a patient's eye can be equal in size, or can be of different sizes. In some embodiments, the visual indicators 310 include physical features, such as holes, depressions, fiducial markings, or the like, formed into ribbons 300. In some embodiments, the visual indicators 310 comprise a dye or pigment disposed at or near ribbon ends.

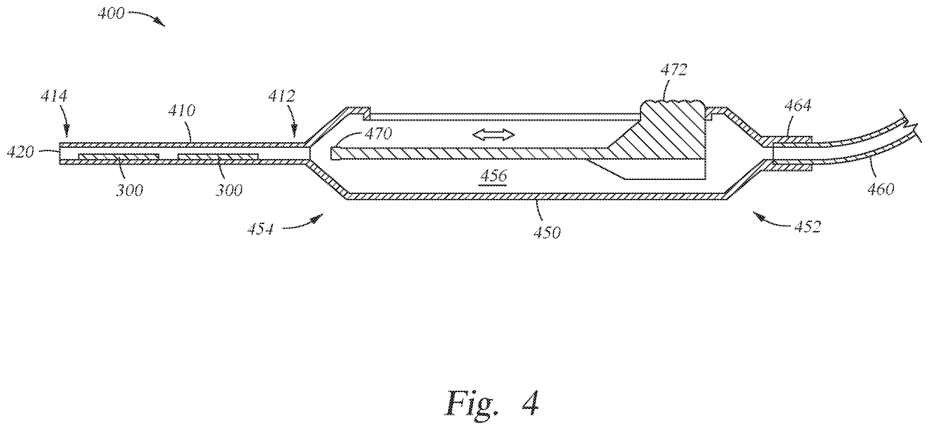

FIG. 4 depicts an applicator 400, according to some embodiments. The applicator 400 may be used to deliver and apply one or more ribbons of biodegradable polymer and retinal cells to a target area within a patient's eye. Generally, the applicator 400 comprises a handle 450 and a cannula 410. The cannula 410 has a proximal end 412 coupled to a distal end 454 of the handle 450, and a distal end 414 opposite the proximal end 412. In some embodiments, a flexible tubing 460 may be disposed through, or coupled to, a proximal end 452 of the handle 450 for supplying vacuum suction to the cannula 410 for aspiration of ribbons. The flexible tubing 460 may be in fluid communication with a vacuum source at a proximal end thereof, and in fluid communication with the cannula 410, either directly or indirectly, at a distal end 464 thereof. For example, the flexible tubing 460 may couple directly to the cannula 410 through the handle 450, or the flexible tubing 460 may couple indirectly to the cannula 410 via an internal chamber 456 of the handle 450. The activation of the vacuum source and thus, the supply of vacuum through the flexible tubing 460 may be controlled by a proportional foot pedal, or toggle on the applicator 400.

The cannula 410, which may comprise a tube, is generally formed of any suitable surgical-grade materials, such a metallic or thermoplastic polymeric materials. Examples of metallic materials include aluminum, stainless steel, and other metallic alloys. Examples of suitable thermoplastic polymeric materials include polyether ether ketone (PEEK), polyetherketone (PEK), and polytetrafluoroethylene (PTFE). In some embodiments, an inner surface of the cannula 410 comprises a surface material selected from a group consisting of fluorinated ethylene propylene, polytetrafluoroethylene, and the like.

In some embodiments, the cannula 410 comprises a rectangular-shaped tube (e.g., having a rectangular cross-section), and or the tube of the cannula 410 comprises a rectangular-shaped distal tip. In some embodiments, the cannula 410 has an opening 420 at the distal end 414 that is rectangular-shaped. The rectangular shape of the cannula 410 and/or opening 420 may facilitate the aspiration and transfer of ribbons with rectangular cross-sectional areas. The rectangular cross-section ribbons disclosed herein can also be aspirated and transferred using conventional cannulas having circular cross-sections.

In some embodiments, the applicator 400 further comprises a plunger 470 disposed through at least a portion of the handle 450 (e.g., a portion of the internal chamber 456) and configured to translate into, and through, the cannula 410. The plunger 470 may be utilized to push out, or dispense, ribbons that have been aspirated into the cannula 410 during a surgical procedure to apply the ribbons to a target area of the eye. In some embodiments, the plunger 470 is attached to a sliding toggle 472 movably coupled with the handle 450, which facilitates manual control of the plunger 470 by a user. In other embodiments, however, the plunger 470 may be electromechanically or pneumatically actuated, e.g., via an electromechanical motor or the supply of fluidic pressure through the flexible tubing 460. In such embodiments, the plunger 470 may be controlled by a foot pedal or other user input device.

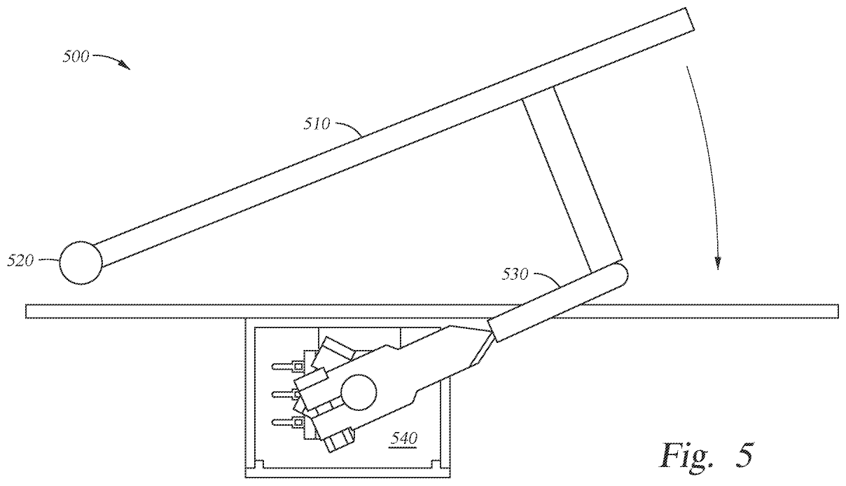

FIG. 5 depicts a proportional pedal controller 500 that can be used in conjunction with an applicator device, such as the applicator 400, to aspirate and deposit ribbons as disclosed herein. Proportional pedal controller 500 includes a foot pedal 510 that is hinged at fulcrum 520. Pedal 510 is coupled to a sensing arm 530 that is connected to a controller unit 540. The proportional pedal controller 500 can be communicatively coupled to the applicator device, and the proportional pedal controller and applicator device can be used in conjunction to aspirate one or more ribbons into a cannula of the applicator device and to deposit the ribbon(s) from the cannula to an appropriate location in a patient's eye. The foot pedal 510 can be coupled to the applicator device through a variety of coupling means, including a hydraulic coupling, a computerized coupling using an electronic sensor one or more servo motors, a direct pressure-transfer coupling, and the like.

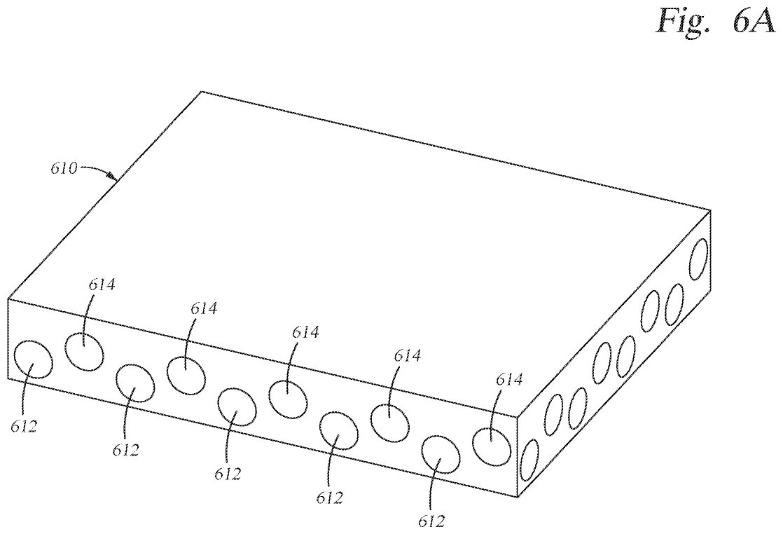

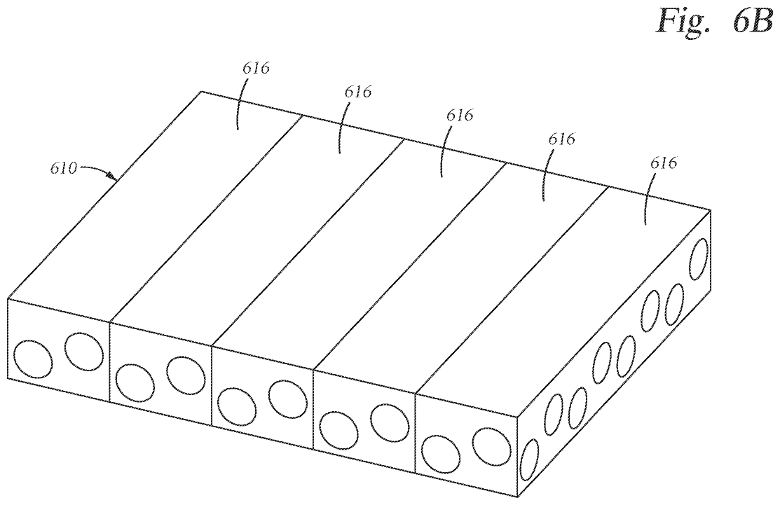

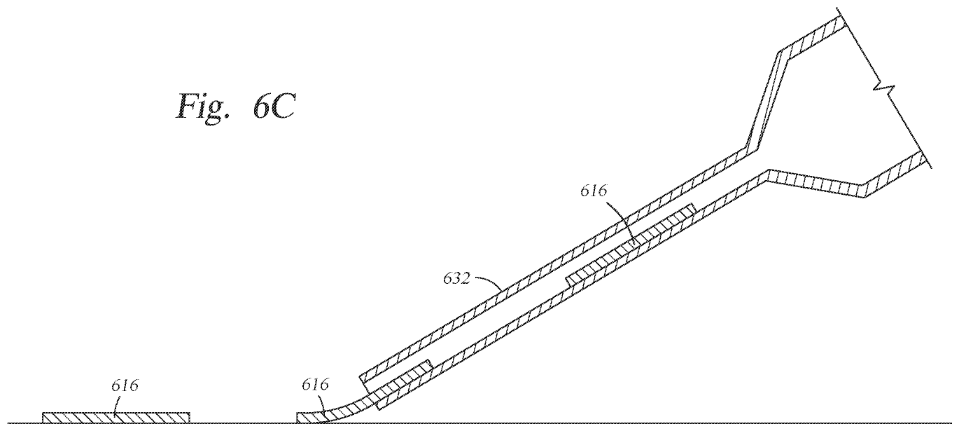

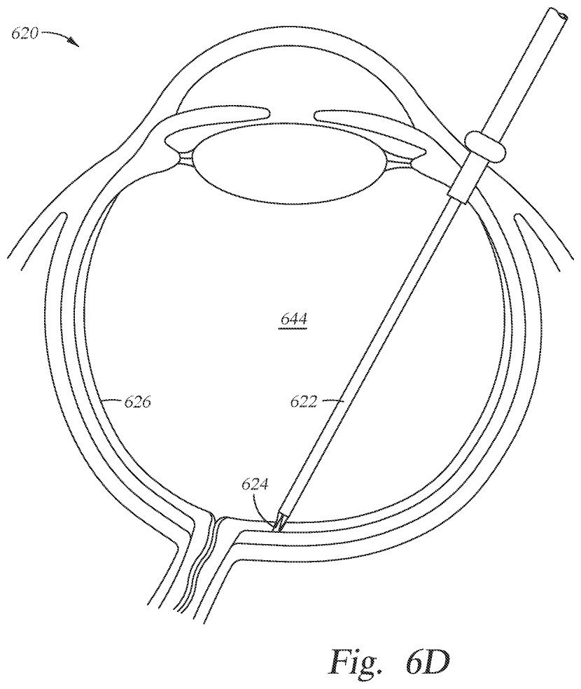

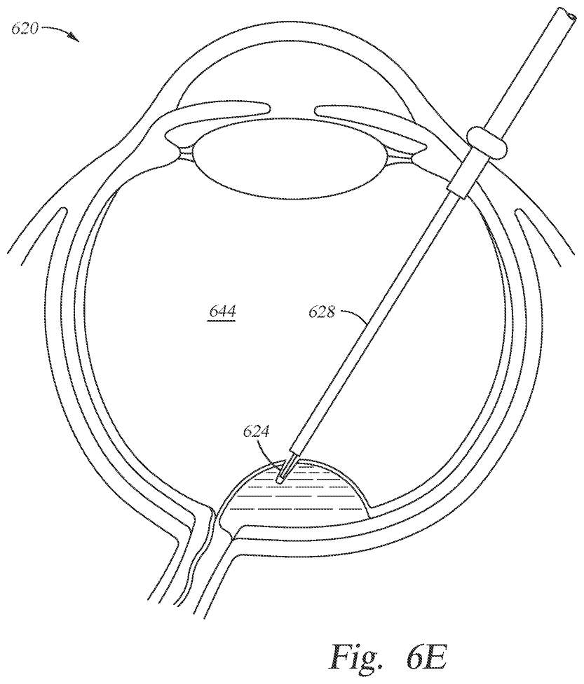

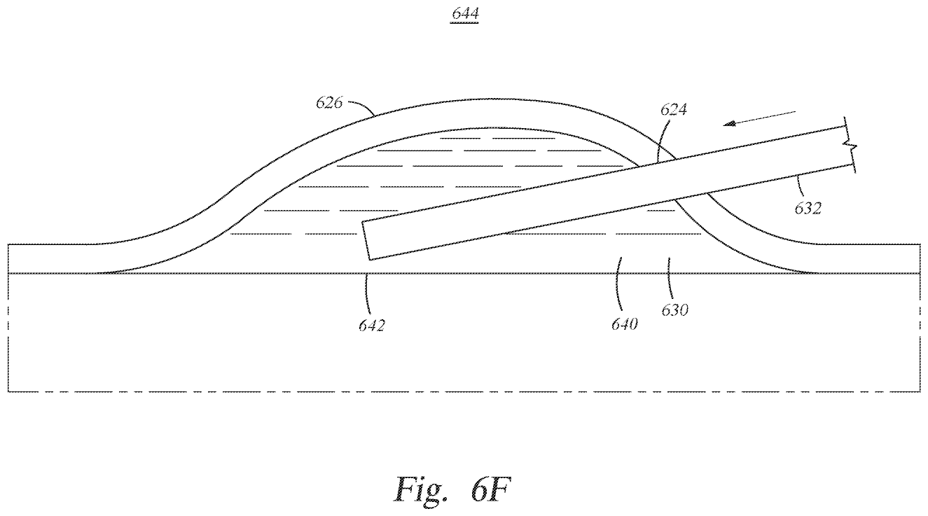

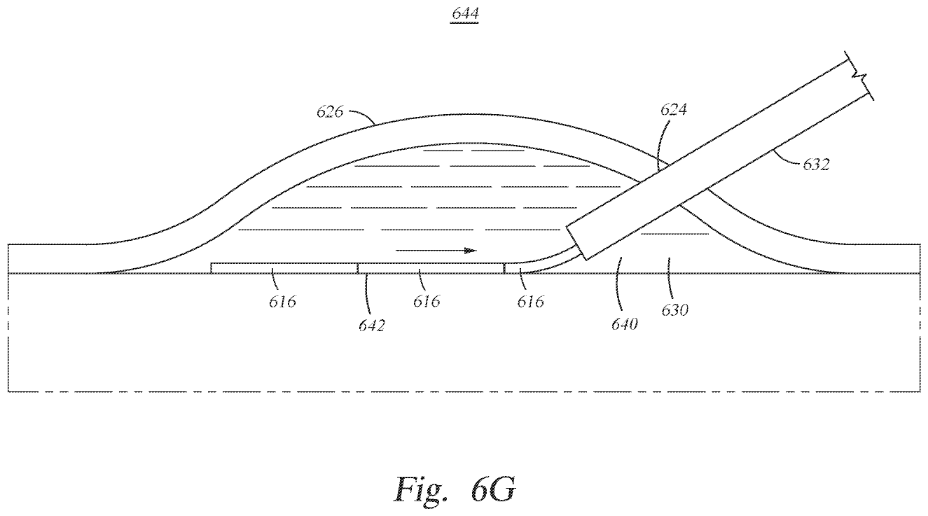

FIGS. 6A-6F depict various operations during an exemplary method for retinal cell replacement, wherein a target area is disposed within or adjacent to the subretinal space. As shown in FIG. 6A, a biodegradable polymer scaffold sheet 610 is embedded with a monolayer or bilayer of retinal pigment epithelial (RPE) cells 612 and retinal photoreceptor progenitor cells 614, which may be derived from induced pluripotent stem cells. The cells are arranged with the RPE cells 612 having a basal polarity and the retinal photoreceptor progenitor cells 614 having an apical polarity. In FIG. 6B, the sheet is cut into a plurality of ribbons 616, and in FIG. 6C, the ribbons 616 are aspirated into a cannula 632 of a ribbon applicator device (e.g., applicator 400) by applying a vacuum suction through cannula 632. In some embodiments, visual indicators included at ribbon ends assist the user in identifying where a ribbon 616 begins and ends.

In FIG. 6D, a scissors or other surgical tool 622 is introduced into an eye 620 of a patient (e.g., via a trocar cannula and sclerotomy) and guided through an intraocular space 644 thereof, and an incision 624 (e.g., a retinotomy) is made in the retina 626 of the eye 620. In FIG. 6E, an injection device 628 is introduced into the eye 620, and a buffered salt solution (e.g., BSS) or viscoelastic is injected through the incision 624 and into a subretinal space 640 to form a bleb 630. In FIG. 6F, after removal of the injection device 628, the cannula 632 of the ribbon applicator device is introduced into the eye 620 and guided though the incision 624 to a far side of the bleb 630. Thereafter, in FIG. 6G, the plurality of ribbons 616 in the cannula 632 are injected from the cannula 632 (e.g., by actuation of a plunger or other injecting mechanism) and over a target area 642 as the cannula 632 is slowly withdrawn from the bleb 630.

In some embodiments, the ribbons 616 are deposited confluently over the target area 642 of the patient's eye. The confluently-deposited ribbons 616 can resemble a stack bond wooden flooring or masonry pattern (e.g., tiling or laid bricks), where the ribbons 616 are deposited side-by-side such that ribbon 616 abuts against and is contiguous with an adjacent ribbon 616.

After the cannula 632 is withdrawn from the eye 620, the process may be repeated one or more times, through the same retinotomy or additional retinotomies, to apply additional ribbons 616. The additional ribbons 616 may be placed adjacent to the previously-deposited ribbons 616, such that the ribbons 616 are positioned in a confluent relationship.

### EXAMPLE EMBODIMENTS

Embodiment 1: A method of generating gelatinous biodegradable ribbons for retinal replacement, comprising: providing a receptacle comprising a space and at least one partition member; delivering a solution of a biodegradable polymer to the space; delivering retinal cells to the space; and culturing in vitro for a period of time said retinal cells and said biodegradable polymer to afford gelatinous ribbons comprising a biodegradable polymer matrix and said retinal cells.

Embodiment 2: The method of Embodiment 1, wherein the at least one partition member divides the space into a first sub-space and a second sub-space.

Embodiment 3: The method of Embodiment 1, wherein an integer number n of partition members divide the space into an integer number n+1 sub-spaces.

Embodiment 4: The method of Embodiment 1, wherein the receptacle comprises substantially vertical interior walls.

Embodiment 5: The method of Embodiment 1, wherein the at least one partition member comprises substantially vertical walls.

Embodiment 6: The method of Embodiment 2, wherein the first and second sub-spaces are cuboid-shaped, with a sub-space length dimension is greater than a sub-space width dimension.

Embodiment 7: The method of Embodiment 3, wherein each of the integer number n+1 sub-spaces is cuboid-shaped, with a sub-space length dimension greater than a sub-space width dimension.

Embodiment 8: The method of Embodiment 2, wherein the first and second sub-spaces each comprise a substantially rectangular cross-sectional area.

Embodiment 9: The method of Embodiment 3, wherein each of the integer number n+1 sub-spaces comprises a substantially rectangular cross-sectional area.

Embodiment 10: The method of Embodiment 1, wherein the retinal cells comprise differentiated cells, progenitor cells, precursor cells, or a combination thereof.

Embodiment 11: The method of Embodiment 1, wherein the retinal cells are selected from a group consisting of retinal pigment epithelial cells, rod cells, cone cells, horizontal cells, bipolar cells, amacrine cells, retinal ganglion cells, and a combination thereof.

Embodiment 12: The method of Embodiment 1, wherein the retinal cells comprise differentiated retinal pigment epithelial cells.

Embodiment 13: The method of Embodiment 1, wherein the retinal cells comprise photoreceptor progenitor cells.

Embodiment 14: The method of Embodiment 13, wherein the photoreceptor progenitor cells comprise rod progenitor cells, cone progenitor cells, or a combination thereof.

Embodiment 15: The method of Embodiment 1, wherein the retinal cells comprise differentiated retinal pigment epithelial cells, rod progenitor cells, and cone progenitor cells.

Embodiment 16: The method of Embodiment 1, wherein the biodegradable polymer comprises a polymer selected from a group consisting of poly(lactic-co-glycolic acid) (PLGA), collagen, gelatin, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, calcium phosphate, and combinations thereof.

Embodiment 17: The method of Embodiment 1, further comprising delivering to the space at least one pharmacological agent, at least one cell nutrient, or a combination thereof.

Embodiment 18: A method of implanting retinal cells into a patient's eye, comprising: aspirating a gelatinous biodegradable ribbon into a cannula, wherein the gelatinous biodegradable ribbon comprises a biodegradable polymer matrix and retinal cells;

forming an incision in a sclera of the patient's eye; inserting the cannula through the incision in the sclera of the eye; and depositing the gelatinous biodegradable ribbon in a target area of the patient's eye.

Embodiment 19: The method of Embodiment 18, wherein the retinal cells comprise differentiated cells, progenitor cells, precursor cells, or a combination thereof.

Embodiment 20: The method of Embodiment 19, wherein the differentiated cells, progenitor cells, precursor cells, or combination thereof comprises retinal pigment epithelial cells, rod cells, cone cells, horizontal cells, bipolar cells, amacrine cells, retinal ganglion cells, or a combination thereof.

Embodiment 21: The method of Embodiment 18, wherein the retinal cells comprise differentiated retinal pigment epithelial cells.

Embodiment 22: The method of Embodiment 18, wherein the retinal cells comprise photoreceptor progenitor cells.

Embodiment 23: The method of Embodiment 22, wherein the photoreceptor progenitor cells are selected from a group consisting of rod progenitor cells, cone progenitor cells, and a combination thereof.

Embodiment 24: The method of Embodiment 18, wherein the retinal cells comprise differentiated retinal pigment epithelial cells, rod progenitor cells, and cone progenitor cells.

Embodiment 25: The method of Embodiment 18, further comprising depositing a double layer of ribbons by: aspirating a second gelatinous biodegradable ribbon into the cannula or a second cannula, wherein the second gelatinous biodegradable ribbon comprises a second biodegradable polymer matrix and additional retinal cells; inserting the cannula or the second cannula through the incision in the sclera of the eye; and depositing the second gelatinous biodegradable ribbon in the target area or a second target area of the patient's eye.

Embodiment 26: The method of Embodiment 25, wherein the double layer of ribbons comprises (1) a first, basal ribbon of the double layer of ribbons comprising differentiated retinal pigment epithelial cells, and (2) a second, apical gel ribbon comprising photoreceptor progenitor cells.

Embodiment 27: The method of Embodiment 18, further comprising forming a plurality of incisions in the retina of the patient's eye, progressively inserting the cannula through each of the plurality of incisions in the retina of the eye, and depositing a plurality of ribbons in a target area of the patient's eye.

Embodiment 28: The method of Embodiment 27, further comprising depositing the ribbons confluently in the target area of the patient's eye.

Embodiment 29: The method of Embodiment 18, wherein the biodegradable polymer matrix comprises a material selected from a group consisting of poly(lactic-co-glycolic acid) (PLGA), collagen, gelatin, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatide, calcium phosphate, and combinations thereof.

Embodiment 30: The method of Embodiment 29, wherein the biodegradable polymer matrix comprises PLGA.

Embodiment 31: The method of Embodiment 18, further comprising depositing the ribbon in a sub-retinal space of the patient's eye.

Embodiment 32: The method of Embodiment 18, wherein the cannula comprises a substantially rectangular cross-sectional area.

Embodiment 33: The method of Embodiment 18, wherein the cannula comprises a surface material selected from a group consisting of fluorinated ethylene propylene, polytetrafluoroethylene, others to be included.

Embodiment 34: The method of Embodiment 18, wherein the ribbon further comprises at least one pharmacological agent, at least one cell nutrient, or a combination thereof.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments may fall within the scope of the appended claims.

What is claimed is:

1. A method of implanting retinal cells into a patient's eye, comprising:
combining retinal cells and a biodegradable polymer scaffold to form a gel;
aspirating at least a portion of the gel into a first cannula;
forming an incision in a sclera of the patient's eye;
inserting the first cannula through the incision in the sclera of the patient's eye; and
depositing the gel in a first target area of the patient's eye.

2. The method of claim 1, wherein the retinal cells are selected from a group consisting of differentiated cells, progenitor cells, precursor cells, and a combination thereof.

3. The method of claim 1, wherein the retinal cells are selected from a group consisting of retinal pigment epithelial cells, rod cells, cone cells, horizontal cells, bipolar cells, amacrine cells, retinal ganglion cells, and a combination thereof.

4. The method of claim 1, wherein the retinal cells comprise differentiated retinal pigment epithelial cells.

5. The method of claim 1, wherein the retinal cells comprise photoreceptor progenitor cells.

6. The method of claim 5, wherein the photoreceptor progenitor cells comprise rod progenitor cells, cone progenitor cells, or a combination thereof.

7. The method of claim 1, wherein the retinal cells comprise differentiated retinal pigment epithelial cells, rod progenitor cells, and cone progenitor cells.

8. The method of claim 1, further comprising depositing a double layer of gels by:
combining additional retinal cells and a second biodegradable polymer scaffold to form a second gel;
aspirating at least a portion of the second gel into the first cannula or a second cannula;
inserting the first cannula or the second cannula through the incision in the sclera of the patient's eye; and
depositing the second gel in the first target area or a second target area of the patient's eye.

9. The method of claim 1, wherein the gel comprises a first basal layer comprising differentiated retinal pigment epithelial cells and a second apical layer comprising photoreceptor progenitor cells.

10. The method of claim 1, further comprising cutting the gel into one or more ribbons prior to aspirating the gel into the first cannula.

11. The method of claim 10, wherein the one or more ribbons have a substantially rectangular cross-sectional area.

12. The method of claim 10, further comprising depositing the one or more ribbons confluently in the first target area or a second target area of the patient's eye.

13. The method of claim 1, wherein the biodegradable polymer scaffold comprises a material selected from a group consisting of poly(lactic-co-glycolic acid) (PLGA), collagen, gelatin, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatide, calcium phosphate, and combinations thereof.

14. The method of claim 13, wherein the biodegradable polymer scaffold comprises PLGA.

15. The method of claim 1, further comprising depositing the gel in a sub-retinal space of the patient's eye.
